(19) United States
(12) Patent Application Publication
(10) Pub. No.: US 2017/0089881 A1
(43) Pub. Date: Mar. 30, 2017

(54) SYSTEM AND METHOD FOR HIGH-THROUGHPUT, OPTOMECHANICAL FLOW CYTOMETRY

(71) Applicant: The Board of Trustees of the University of Illinois, Urbana, IL (US)

(72) Inventors: Gaurav Bahl, Champaign, IL (US); Kewen Han, Champaign, IL (US)

(21) Appl. No.: 15/278,581

(22) Filed: Sep. 28, 2016

Related U.S. Application Data
(60) Provisional application No. 62/234,178, filed on Sep. 29, 2015.

(51) Int. Cl. G01N 33/487; G01N 29/02; G01N 29/24; G01N 15/14; G01N 21/17

(52) U.S. Cl. CPC G01N 33/487; G01N 15/1436; G01N 21/1702; G01N 29/2418; G01N 29/02; G01N 2015/0065

(57) ABSTRACT

A system and method includes resonator device to detect cells or other particles through light and/or vibration sensing.

### CROSS REFERENCE TO RELATED APPLICATION

This application claims the benefit of U.S. Provisional application Ser. No. 62/234,178, filed on Sep. 29, 2015, the disclosure of which is incorporated by reference herein in its entirety.

### GOVERNMENT LICENSE RIGHTS

This invention was made with government support under ECCS-1408539 awarded by the National Science Foundation. The government has certain rights in the invention.

### BACKGROUND

Flow cytometry is a laser-based, biophysical technology employed in cell counting, cell sorting, biomarker detection and protein engineering, by suspending cells in a stream of fluid and passing them by an electronic detection apparatus. Flow cytometry can allow analysis of the physical and/or chemical characteristics of many thousands of particles per second. Flow cytometry is used in the diagnosis of health disorders, including blood cancers, and other applications in research, clinical practice and clinical trials. A variation of flow cytometry is to physically sort particles based on their properties, so as to purify particle groups of interest.

### BRIEF DESCRIPTION OF THE DRAWINGS

In association with the following detailed description, reference is made to the accompanying drawings, where like numerals in different figures can refer to the same element.

FIG. 1A-E are diagrams of an example resonator device.

FIG. 2 is a block diagram of example acoustic whispering-gallery mode (WGM) frequencies determined by momentum conservation.

FIGS. 3A-F are graphs of example observation of mechanical oscillations in water-filled devices.

FIGS. 4A and 4B illustrate example determined equatorial mechanical mode profiles.

FIGS. 5A-E are example graphs characterizations of 99 MHz acoustic WGM in a water-filled device.

FIG. 6 is an example graph of an optomechanical sensing of sucrose solutions.

FIG. 7 is a system diagram of an example opto-mechano-fluidic resonator platform.

FIG. 8 is a picture of an example silica resonator device and tapered waveguide fibers.

FIG. 9 is a diagram of an example mechanism of radiation pressure based optomechanical actuation of the resonator device and the resulting optical signals that are generated.

FIG. 10 is a perspective view of a sample vibration mode of the resonator device.

FIG. 11 is a cutaway view of an example opto-mechanical sensing of fluids and fluid suspended flowing particles in the resonator device.

FIG. 12 is a schematic illustrating an example real-time high-throughput sensing.

FIG. 13 is graphs illustrating an example real-time high-throughput sensing.

FIGS. 14A-D are graphs illustrating an example real-time high-throughput sensing.

FIG. 15 is a graph of example particle location according to an acoustic mode topology particle sensing mechanism.

FIG. 16 is a block diagram illustrating an example optomechanical-fluidic physics.

### DETAILED DESCRIPTION

Cells can exhibit unique mechanical signatures corresponding to their diseased or healthy states, but current methods to analyze large cell populations (e.g. through flow cytometers) are limited since they only perform optical measurements on single cells. The potential use of optomechanical devices, that support coupled vibrational and optical-modes, could allow optical measurements of the mechanical signatures of individual particles such as cells. While optomechanics is presently used in a broad variety of solid-state systems, there have been very few optomechanical systems that operate with liquids. One key issue is that the direct liquid immersion of optomechanical systems is challenging since the vibrational energy simply leaks out to the higher-impedance liquid surrounding the device.

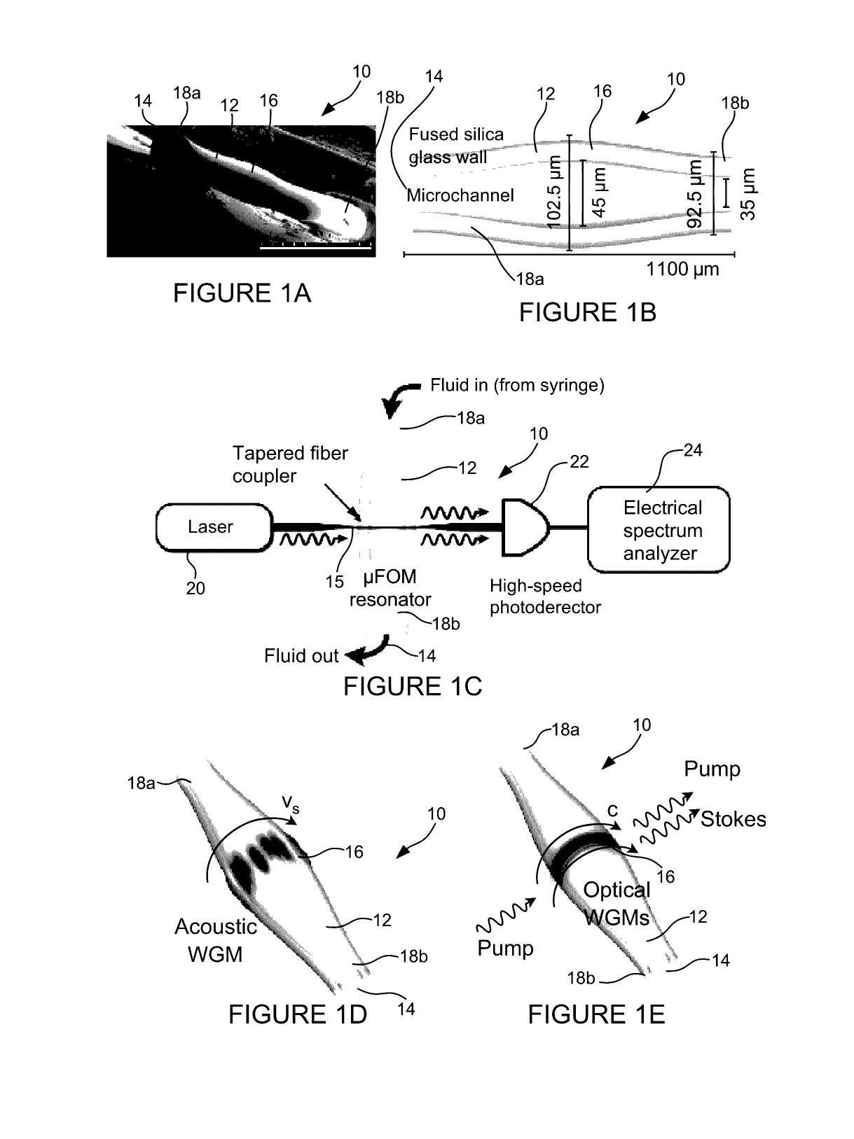

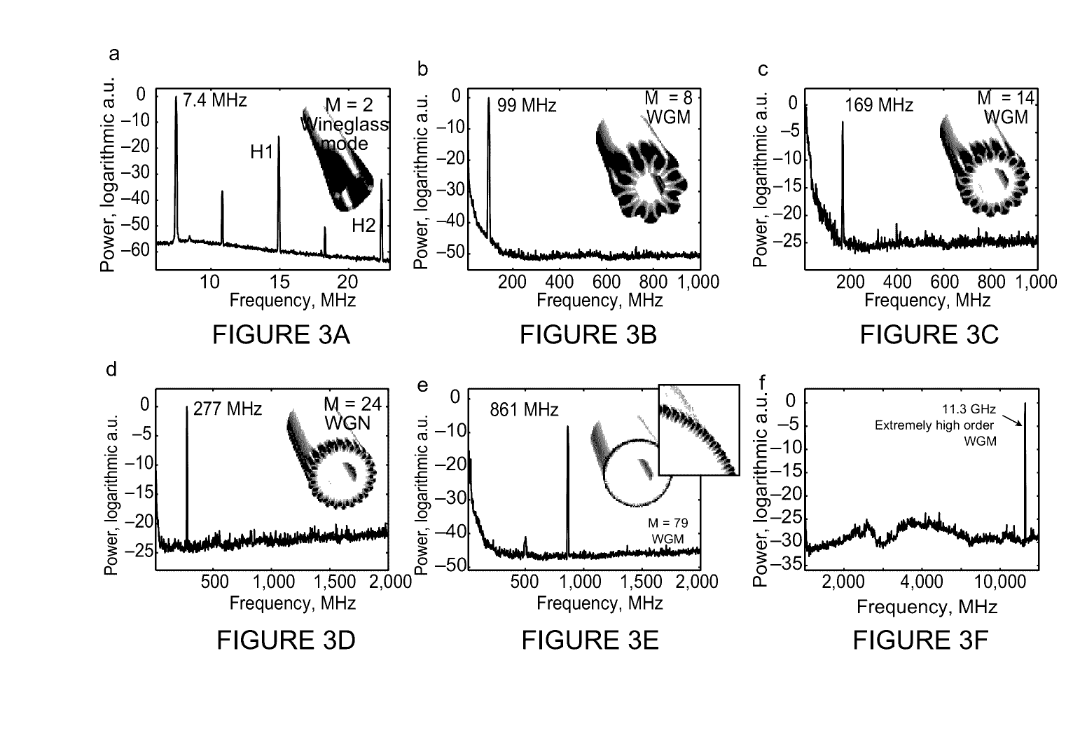

FIG. 1A-E are diagrams of an example resonator device 10, e.g., a microfluidic optomechanical fluidic resonator (OMFR) device, which can enable real-time, high-throughput, optomechanical flow cytometry. FIG. 1A illustrates a colorized scanning electron micrograph of the resonator device 10, also known as a resonator, showing the local bulges in the as a function of position (scale bar indicates 200 m). In some embodiments, the resonator device 10 is constructed of fused silica glass walls 12 forming a channel 14. In some embodiments, the walls 12 are generally cylindrical shaped. In some embodiments, a configuration of the resonator device 10 can include a center portion 16 that is wider in diameter than tapered end portions 18a, b. FIG. 1B illustrates example dimensions of the resonator device 10 used to obtain mechanical wineglass modes in FIG. 3A, and mechanical whispering-gallery modes in FIG. 1D and FIG. 3B-F, and mechanical radial modes in FIG. 9 and FIG. 10, e.g., a channel 14 diameter of 35 micrometers at the narrow ends 18a, b and 45 micrometers at a widest point of the center portion 16, and a resonator device 10 outer diameter of 92.5 micrometers at the narrow ends 18a, b and 102.5 at the widest point of the center portion 16. Other dimensions can be used. FIG. 1C illustrates an example tapered optical fiber 15 used to couple light in and out of the whispering-gallery optical modes of the resonator device 10. Mechanical vibrations of the resonator device are generated by either thermal fluctuations, or through applied optical radiation pressure, or though various forms of Brillouin scattering, and other optical forces 10. In some examples, the systems and methods can employ a telecom wavelength 1.5 micron wavelength pump laser, or other light source 20, without any modulation applied. A high-speed photodetector 22 generates an electrical current corresponding to the mechanical vibration spectrum by measuring scattering of light from the resonator, e.g., the beat note between the optical pump and the scattered light. The optical signals can be measured by an electrical spectrum analyzer 24 that measures the output electrical current from the photodetector 22. An example configuration for forward light scattering is illustrated in FIG. 1C. FIG. 1D illustrates a mechanical whispering-gallery mode on the resonator device 10, showing that mechanical displacement as concentrated at the equator. The mode circulates at the velocity of sound, vₛ. FIG. 1E illustrates the optical whispering-gallery modes of the resonator device 10 that travels at the speed of light, c. In some embodiments an optical signal can extract a mechanical signature for measurement and analysis, and/or a mechanical vibration provides a mechanical signature or an optical signal provides an optical signature, etc.

In some embodiments, the resonator device 10 can confine liquids within channel 14 and optical observation of various mechanical modes at frequencies ranging from 2 MHz to 11,000 MHz enables measurement of the mechanical properties of the liquid. In one example, a mechanical mode with Q=4700 can be observed at 99 MHz in a water-filled resonator device 10. Multiple mechanical modes can be used to modulate the light to extract more information on the liquid inside the resonator. Vibrations are sustained even for very high viscosity liquids, including test liquids with higher viscosity than that of blood. The resonator device 10 can enable optomechanical investigation with liquids, while light is conventionally coupled from the outer dry side of the capillary, and liquids are provided by a standard microfluidic inlet, e.g., syringe. In some examples, real-time label-free acoustic sensing (weighing, moduli, sizing) of cells at rates up to 10,000 cells/second may be achieved, e.g., without the use of tagging or binding agent, etc.

In FIGS. 1A, B the resonator device 10 can be fabricated from a lengthwise-drawn fused silica capillary with its radius modulated as a function of length, an example of which is described below. At the widest point 26, resonator devices 10 forms a microresonator that resonantly enhances both light and vibration (FIG. 1D, E), simultaneously, while supporting a considerable overlap between the optical and mechanical resonant modes. The acoustic modes can also be optically excited by means of forward and backward stimulated Brillouin scattering, e.g., F-SBS and B-SBS respectively.

Stimulated Brillouin scattering is an optical gain mechanism for laser light sources 20 and for nonlinear optics, and is also used for optical phase conjugation and slow light. All materials support Brillouin scattering, including structures like bulk materials and fibers, to droplets, nano-spheres, photonic-crystal fibers and crystalline resonators. The recent demonstration of Brillouin scattering in microspheres was followed by the Brillouin cooling and excitation of their vibrational modes as well; which indicates that Brillouin effects can serve as a general mechanism for actuating (and interrogating) vibration in various types of micro-mechanical resonators. In separate research on optofluidic devices, the motion of liquids can be used to control light, but light has rarely been used to actuate a fluid.

The Brillouin scattering of light from vibration (or sound) to excite and measure vibration in the resonator device 10, e.g., can be used to examine cells. The resonator devices 10 can exhibit mechanical deformation at their solid-fluid interface. Confinement of the test fluid inside the device reduces the acoustic loss. The cells may be optionally treated with fluorescent molecules that are bound to determined proteins and launched through the resonator device 10 while light is shined through the cells to look at the fluorescence, e.g., via forward scatter, back scatter and/or side scatter, etc. Based on the color of the fluorescence properties of the cells can be determined, e.g., a membrane protein is present on the cell and the cell was treated with a determined drug, etc., to analyze treatment correlations, drug discovery, disease discovery, etc. Additionally or alternatively, the resonator devices 10 can also be used to determine acoustic properties of the cells, e.g., density, stiffness, compressibility, etc., e.g. In this way, the resonator device 10 can provide high-throughput analysis of the cells, or other particles, e.g., on the order of thousands of particles per second. The particle can be of a micro and/or nano-size scale for example.

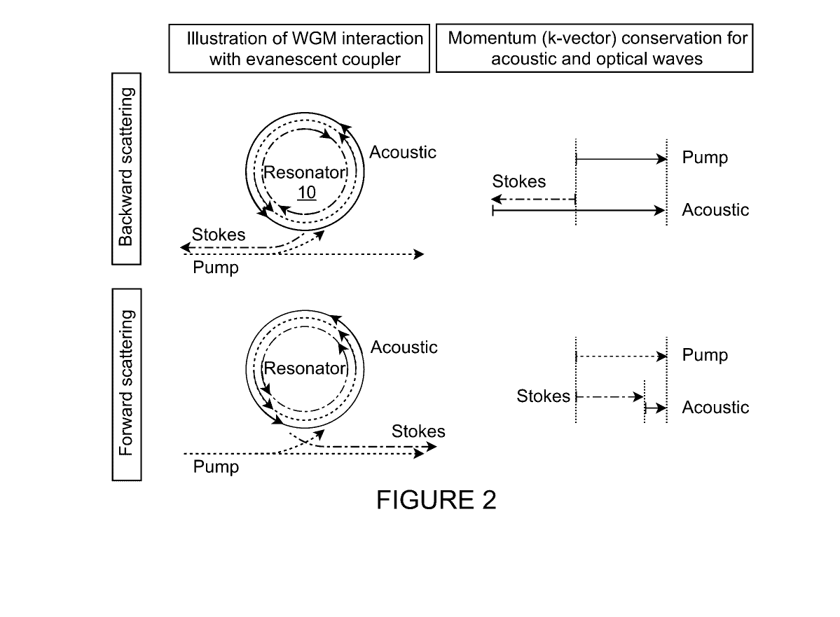

FIG. 2 is a block diagram of example acoustic whispering-gallery mode (WGM) frequencies that are generally determined by momentum conservation. Conservation of momentum between photons and phonons dictates the acoustic WGM frequencies that are generated through both F-SBS and B-SBS. Light traveling in the tapered resonator device 10 couples to optical WGMs of the resonator, and is scattered to lower (Stokes) frequencies in either the forward or backward direction. In the case of back-scattering (B-SBS) momentum conservation enforces long acoustic k-vectors such that the acoustic modes are at high frequencies, e.g. 10 GHz regime. Stokes light is received from the same tapered coupler in the backward direction. In forward scattering (F-SBS) momentum conservation enforces that the acoustic modes are at much lower frequencies (e.g. <1 GHz regime). Stokes light is received in the forward direction as illustrated in FIG. 1C.

FIGS. 3A-F are graphs of example observation of mechanical oscillations in water-filled resonator devices 10. FIG. 3A illustrates wineglass mechanical mode oscillation at 7.4 MHz. H1 and H2 are the first and second harmonics of the mode respectively. Peaks at 10.81 MHz and 18.27 MHz are caused by other mechanical modes of vibration being actuated. FIG. 3B-E illustrate acoustic whispering-gallery mode oscillations of various azimuthal mode orders identified with the number M. These modes are excited by forward stimulated Brillouin scattering e.g. F-SBS. FIG. 3F illustrates very high order acoustic WGM oscillations near 11 GHz are excited by backward stimulated Brillouin scattering e.g. B-SBS.

In the case of B-SBS, the incoming pump light (frequency ωₚ) photoelastically back-scatters from a high-frequency acoustic wave (frequency Ω) in the material to generate a lower frequency Stokes optical signal (frequency ωₛ=ωₚ−Ω), as shown in FIG. 2. Simultaneously, electrostrictive pressure is generated by the two optical signals which amplifies the acoustic wave. Optical spectrum analysis reveals the optical pump and Stokes signals, as described previously. The resulting beat note between pump and Stokes signals measured on an electrical spectrum analyzer is representative of the high-frequency acoustic wave. This B-SBS positive feedback process occurs near Ω=11 GHz mechanical frequency in silica when 1.5 micron wavelength pump light is used. An example measurement of such a mode in this study is shown in FIG. 3F.

The case for forward scattering, e.g. F-SBS, is as described above, except that the pump light photoelastically scatters in the forward direction such that the Stokes signal co-propagates with the pump. This reversal of the scattering direction (to forward) suggests that much smaller phonon momentum is required to bridge between the pump and Stokes momentum gap (FIG. 2). As a result, lower acoustic frequencies (< GHz) are more likely for the F-SBS process. Additional details on the energy and momentum conservation requirements for F-SBS and B-SBS in a circular resonator can be provided. For example, for the capillary geometry, a wide variety of acoustic whispering-gallery modes exist in the shell-type geometry.

Excitation of acoustic whispering-gallery modes in resonator devices 10: The acoustic whispering-gallery mode (WGM) oscillations can be excited in a water-filled resonator device 10, ranging in frequency from 99 MHz to 11,000 MHz (FIGS. 3B-F). As described in FIG. 2, the “Brillouin” optomechanical process allows for excitation and measurement of these acoustic WGM oscillations by coupling continuous-in-time light at an optical resonance, while interrogating the optomechanical oscillation at the fiber output of the system. No modulation of the pump laser is needed. The resonator device 10 can be used to introduce and extract light from the optical WGMs by means of evanescent coupling with a tapered optical fiber 15 (as shown in FIG. 1c). Optical ring-down measurements indicate optical quality factors in excess of Qₒ=10⁸ (example highest measured Qₒ=1.6×10⁸). No contact between the fiber and the resonator device 10 is required for mechanical transduction, so the acoustic quality factors are not degraded. However, contact between the fiber and resonator devices is also permissible. The same optical fiber 15 taper is used to extract the Stokes scattered light from the device, which is then used as a measure of the mechanical vibration within the structure. The example measurements are performed at room temperature and atmospheric pressure, and various solutions are introduced into the device by a syringe pump.

The mechanical modes of the resonator device 10 can be simulated or calculated using 3D finite element modeling using computer software, e.g. Ansys or Comsol multiphysics, as described. Only the glass capillary is modeled, without a liquid, to extract the mechanical modes of the device that correspond to the observed oscillation. Each of these modes is identified with a M-number which is the azimuthal mode order quantifying the number of acoustic wavelengths around the equatorial circumference of the device. In addition, a M=2 whispering-gallery mode at 7 MHz is observed (FIG. 3A), that is generally referred to as a wineglass mode. The harmonics in this wineglass mode oscillation (H1, H2 in FIG. 3A) are typical for such low frequency modes and result from the fact that this is a standing-wave type vibration excited by centrifugal radiation pressure, as opposed to the traveling wave vibrations in FIG. 3B-F. The light and vibration excitations of the resonator can interact together through radiation pressure, optical electrostriction, and optical scattering, such that the light and vibration modes can influence each other.

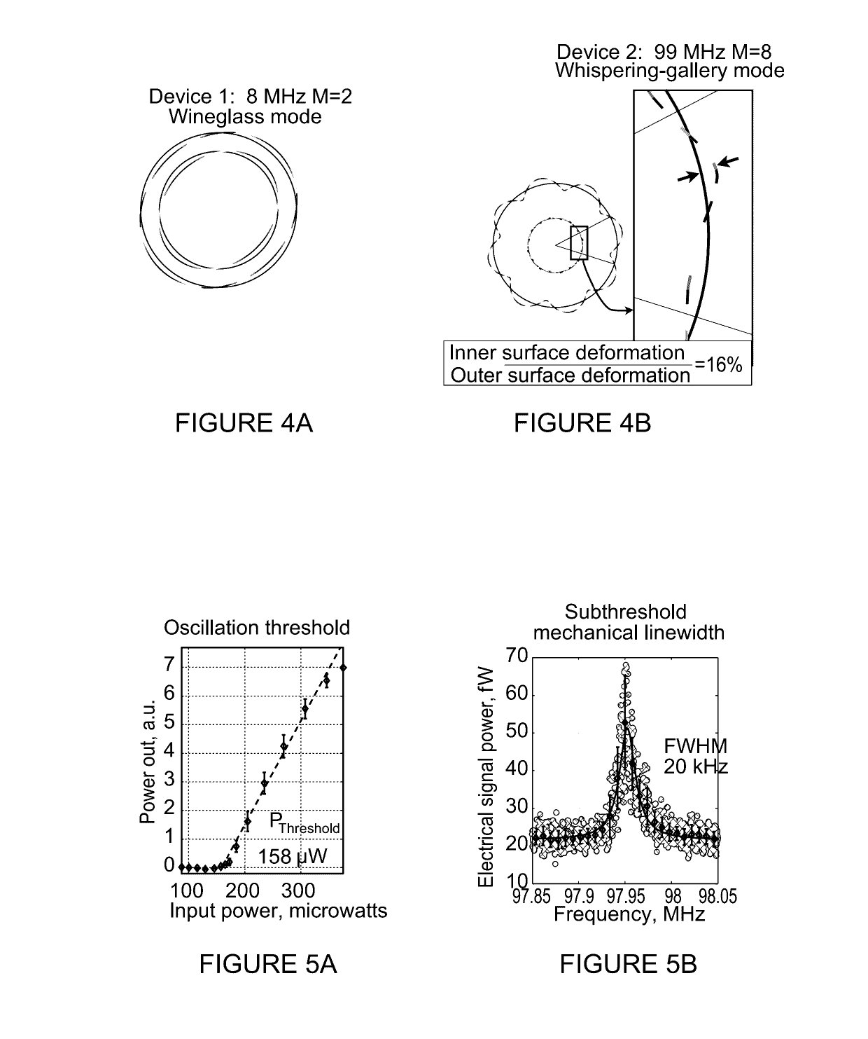

FIGS. 4A and 4B illustrate example simulated equatorial mechanical mode profiles. In FIG. 4A, for the M=2 wineglass mode at 8 MHz (for example appears at 7.4 MHz, see FIG. 3A) the displacement ratio of internal and external surfaces is almost 1:1 at the equator. In FIG. 4B, for the example observed M=8 acoustic WGM at 99 MHz (see FIG. 3B, the inner surface experiences 16% displacement relative to the outer surface.

Quantifying solid-liquid interaction: The penetration of vibration into the liquid scales with the ratio between the acoustic wavelength and the thickness of the resonator wall. Therefore the 7.4 MHz (M=2) mode is calculated to have large penetration into the fluid (FIG. 4A). Finite element calculation shows that the 99 MHz (M=8) mode has a 16% penetration to the fluid at the surface, defined by the deformation amplitude at the inner fluid interface divided by the maximum deformation (FIG. 4B). An arguably better method of quantifying the efficiency of interaction with the liquid would be to calculate the ratio of energy stored in fluid-deformation to the energy stored in the glass-deformation. As this requires modeling of the fluid as well, this preferred quantification method is not chosen here. The higher M modes have lower penetration into the liquid. However, as the resonator can be fabricated with walls as thin as 560 nm a large acoustic penetration to water can be possible in such thin-wall silica bubble resonators even for the 11 GHz mode (M˜650), which can open a rare hypersonic window for the acoustic analysis of liquids. Reduction in wall thickness may even allow access to an interesting regime where giant-enhanced Brillouin scattering is predicted. Alternatively or additionally, radial mechanical modes e.g. FIG. 9 and FIG. 10, have complete penetration into the fluid and enable mechanical sensing to a much greater depth inside the resonator.

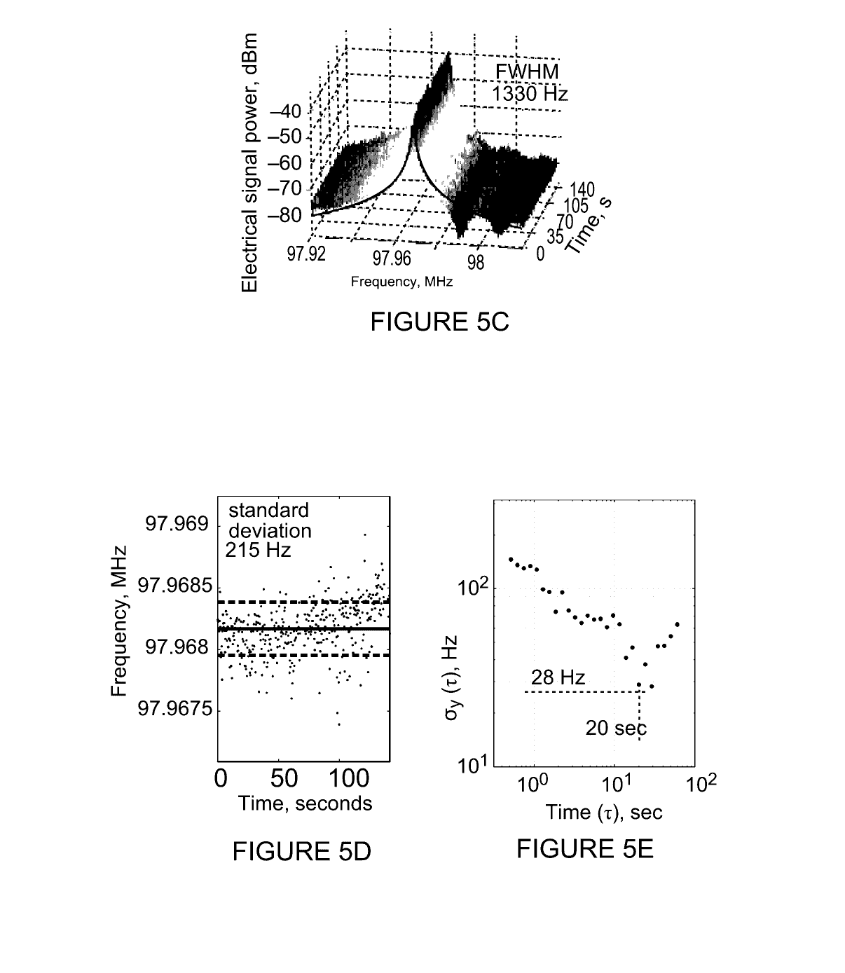

FIGS. 5A-E are example graphs for characterizations of an example 99 MHz acoustic WGM in a water-filled device. In FIG. 5A, a lowest observed optical threshold power to excite mechanical oscillation is 158 microwatts. Error bars indicate variance of each averaged measurement. In FIG. 5B, the intrinsic mechanical linewidth (mechanical quality factor) is directly observed at very low input optical power. Error bars indicate local variance of the averaged raw data. In FIG. 5C, a spectrogram of the mechanical oscillation frequency spectrum is obtained with above-threshold input optical power, where the oscillation is excited using optical forces. In that figure, X-axis is mechanical frequency measured through the beat note of the pump and the vibration-scattered Stokes light on a photodetector, Y-axis is logarithmic mechanical oscillation power, and depth axis is time. In this example the laser light source 20 was rapidly scanned back-and-forth through the region of the optical mode that exhibits phase-matching for this mechanical oscillation. In FIG. 5D, peak frequencies extracted from spectrogram of (c) show a frequency standard deviation of 215 Hz. In FIG. 5E, Allan deviation plot of the frequency data in (d) provides an estimate of the potential sensor resolution.

Characterization of mechanical mode: We measure that the minimum power required to excite these oscillations in the water-filled resonator devices 10 is 158 microwatts (FIG. 5A). Also, a mechanical quality factor Qₘ=4700 can be measured for the 99 MHz (M=8) mode in this water-filled device (FIG. 5B), via the sub-threshold oscillation linewidth, which implies Qₘ×Frequency=4.6×10¹¹. This “f-Q product” compares well against the previously measured Qₘ=12300 in a solid silica sphere for a 95 MHz mode.

This 99 MHz (M=8) mode of vibration can be sustained as long as the CW input laser power is provided. A spectrogram demonstrating this stability (FIG. 5C) is obtained over 140 seconds while periodically scanning through the optical resonance that excites this mechanical mode. While such oscillation data is typically captured with the laser light source 20 at a fixed frequency, it can be practically found that scanning the pump laser light source 20 through the optical resonance is more repeatable. This is because scanning is less affected by unknown drifts. The peak oscillation frequencies (FIG. 5D) can be detected in this spectrogram by means of Lorentzian curve fits (FIG. 5C) at each time-point. The standard deviation of the peak frequency is calculated to be 215 Hz along this 140-second period.

In order to better characterize the stability of the oscillation and the ability of this system to function as a sensor we calculate the Allan deviation (square root of the two-point variance) of the frequency data, which charts the frequency deviation as a function of averaging time (FIG. 5E). It is seen that short-term and long-term frequency drifts dominate device performance. For instance, the long-term upward drift in FIG. 5D manifests as an up-turn in the Allan Deviation data (FIG. 5E) for long averaging times. The short-term frequency instabilities manifest as a large deviation for short averaging times. A “sweet spot” occurs in between, indicating that a 20-second moving average of the sensor output can give a best resolution.

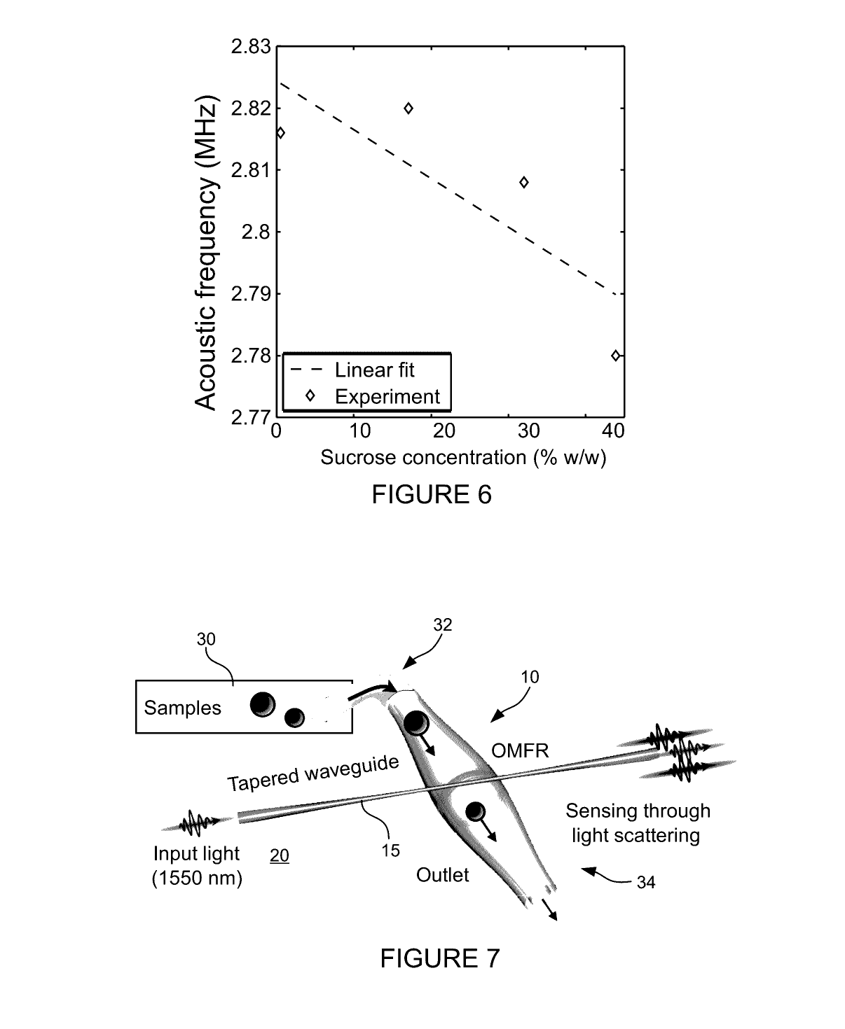

FIG. 6 is an example graph of an optomechanical sensing of liquid sucrose solutions. For example, we measure the frequency of a 2.8 MHz wineglass acoustic mode (M=2) while the concentration of sucrose solution inside the resonator device 10 is varied. The size of the markers in FIG. 6 is approximately the standard deviation (better than 1 kHz) of each frequency measurement. The dashed red line is a linear fit to the data, which indicates a downward trend consistent with increasing effective mass of the resonator. Resonator outer diameter is 184 microns.

Operation with high-density high-viscosity liquids: An example can be performed to prove that the opto-mechanical interaction can be sustained even when the motional mass is high (e.g. high density liquid) and when the fluid-related acoustic energy losses are high (e.g. high viscosity). Sucrose (table sugar) can be incorporated into an aqueous solution inside the resonator and measure the sensitivity of the optomechanically actuated wineglass mode to the sucrose concentration of the fluid. Optomechanical oscillation can be observed at all tested sucrose concentrations. The resulting relationship between acoustic frequency and the sucrose concentration exhibits a non-monotonic trend, which we plot in FIG. 6 along with a linear fit to the data. The net decreasing frequency trend is consistent with increasing effective mass of the oscillator as the fluid density increases. The visibly non-monotonic nature of the trend, however, might originate from non-perturbative high sugar concentrations resulting in nonlinear change in frequency. For a complete model one should consider the change in the fluid properties with the sucrose concentration, including the change of speed of sound. Note that at the highest sucrose concentration that is tested in this example, the solution has a viscosity that is approximately 3-times greater than that of blood. The actuation of optomechanical oscillation with such a high-viscosity liquid opens up the possibility for investigating the acoustic properties of viscous bio-analytes.

In view of the above, a bridge between optomechanics and microfluidics can be demonstrated by confining various liquids inside a hollow microfluidic optomechanical resonator. For example, the optomechanical interaction in the resonator devices 10 is dependent on the fluid contained within. These results are therefore a step towards examples of probing optomechanics on non-solid phases of matter. For example, the high frequency, high quality-factor mechanical modes demonstrated in this work may enable strongly localized, high-sensitivity, optomechanical interaction with chemical and biological analytes since environmental parameters like pressure and cell nutrients are relatively easy to control inside the hollow resonator, and since the liquid volume within the device is the scale of a living cell.

In contrast to other optomechanical systems that typically involve standing-wave mechanical vibrations, here circulating phonons include the acoustic WGMs, which are essentially acoustic vortices. These traveling-wave type acoustic modes carry angular momentum, which creates possibility of using resonator devices 10 to enable optomechanical interactions with vortices in various liquids and condensates.

Example Microfluidic device fabrication: The resonator devices 10 (FIG. 1A,B) can be fabricated with a fused-silica glass capillary that is pulled lengthwise using linear actuators, while being heated with infrared lasers (CO² at 10.6 micron wavelength). By controlling the power of the infrared lasers in the process of pulling, the device diameter can be controlled along its length. Optical WGMs as well as acoustic WGMs are simultaneously well-confined in the regions of large diameter sandwiched between regions of narrow diameter (FIG. 1D, E), enabling a high-degree of modal overlap. Therefore, these large diameter regions form the resonator devices 10, and multiple such resonators can be built on a single capillary.

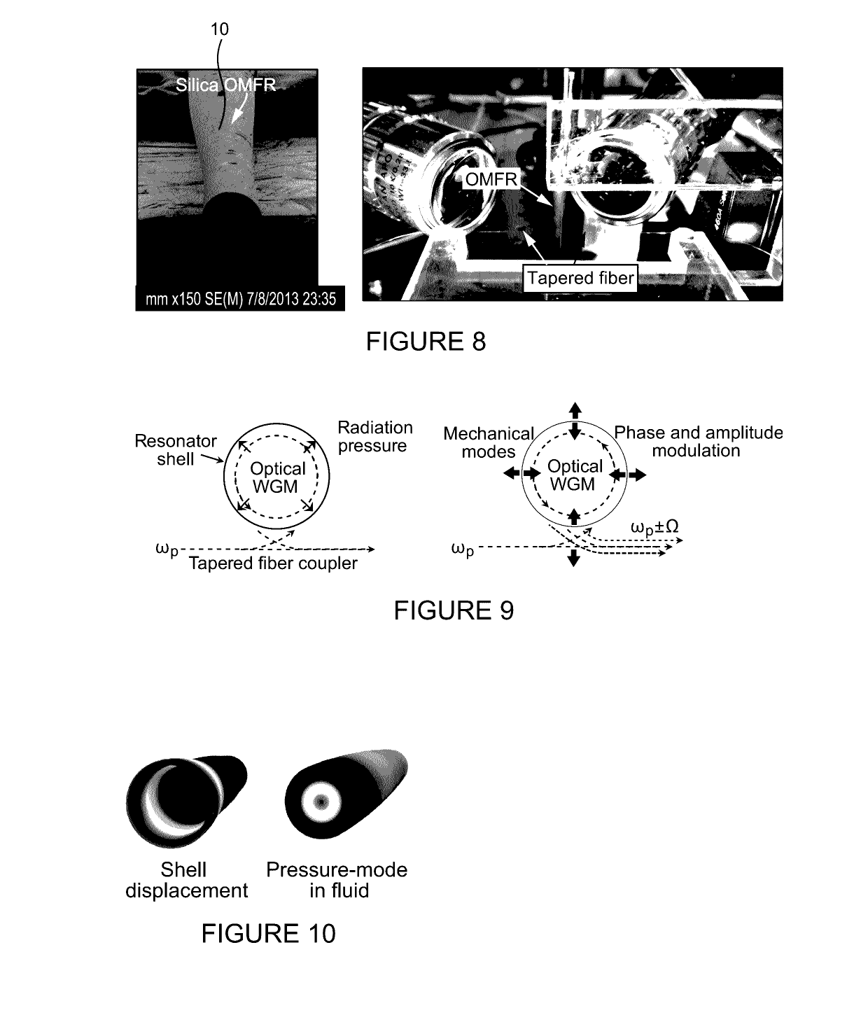

Example method: The resonator device 10 is placed in close proximity (<1 micron distance) to tapered optical fiber 15 (e.g., as shown in FIG. 1C, FIG. 6, and FIG. 8), such that evanescent coupling from the taper allows light to be introduced and extracted from the optical WGMs of the device. A CW fiber-coupled tunable 1.5 micron wavelength laser is used as the light source 20, and a fiber-coupled high-speed photodetector is used to monitor the scattered light. Mechanical vibration is stimulated through optical forces or thermal fluctuations. The generated scattered optical signals are also collected by the tapered fiber 15, and the temporal interference of these signals against the partially transmitted pump laser is used to infer the mechanical response using the current output from the photodetector. An optical spectrum analyzer is used to verify that 4-wave mixing and Raman scattering are not responsible for the beat note observed in the electrical spectrum.

FIG. 7 is a system diagram of an example sensing application. The resonator device 10 obtains samples 30, including suspended particles, from tubing to an input port 32 and directs the sample 30 to an outlet 34. A tapered waveguide can receive an input light of about 1550 nm and the mechanical properties of the sample can be sensed at the output of the waveguide through the light scattering that occurs because of the optomechanical coupling.

FIG. 8 is a picture of an example silica resonator device 10 and tapered waveguide fiber 15. Optical WGMs and acoustic WGMs are simultaneously well-confined in the regions of large diameter sandwiched between regions of narrow diameter, enabling a high-degree of modal overlap.

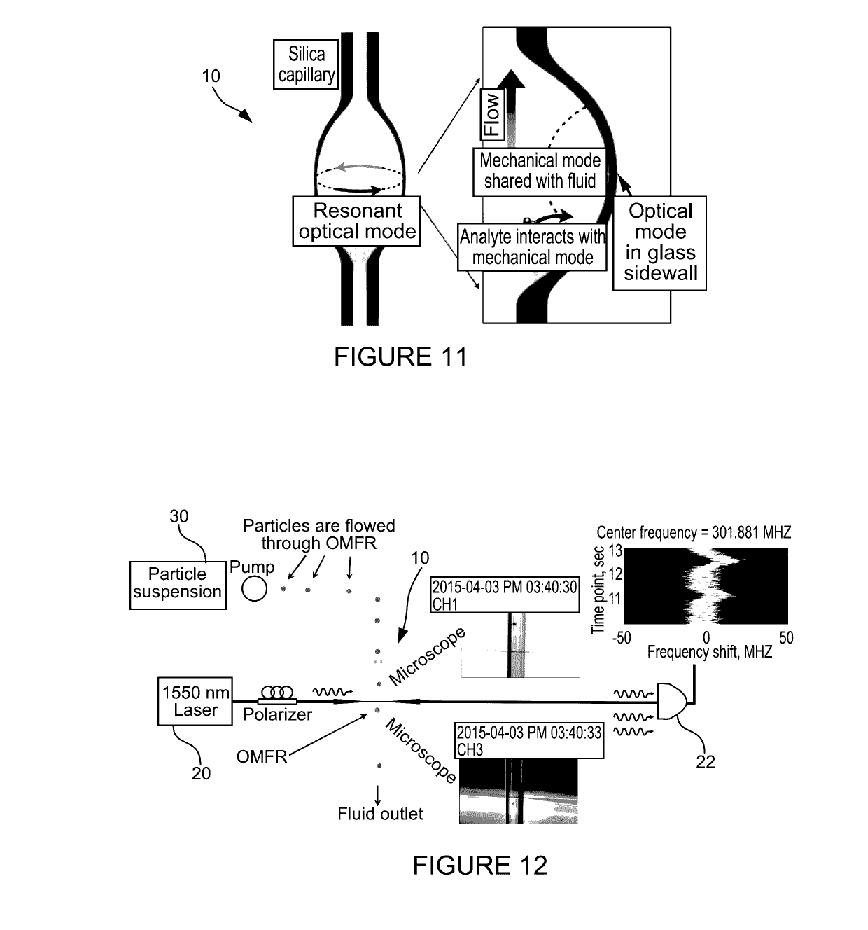

FIG. 9 is a diagram of an example mechanism of radiation pressure based optomechanical actuation of the resonator device 10 and the resulting optical signals that are generated. FIG. 10 is a perspective view of a sample radial vibration mode of the resonator device 10. FIG. 11 is a cutaway view of an example opto-mechanical sensing of fluids and fluid suspended flowing particles in the resonator device 10. In FIG. 9, the left illustrates confinement of light in optical WGMS amplifies radiation pressure. In FIG. 9, the right illustrates Brownian and induced vibration modulates the input light, resulting in sidebands. In FIGS. 10 and 11, hybrid fluid-shell breathing vibrational modes are used for sensing. Fluid motion is entrained by the shell oscillations. Particles in the fluid perturb this hybrid mode. An example vibration frequency is about 30 to about 50 MHz.

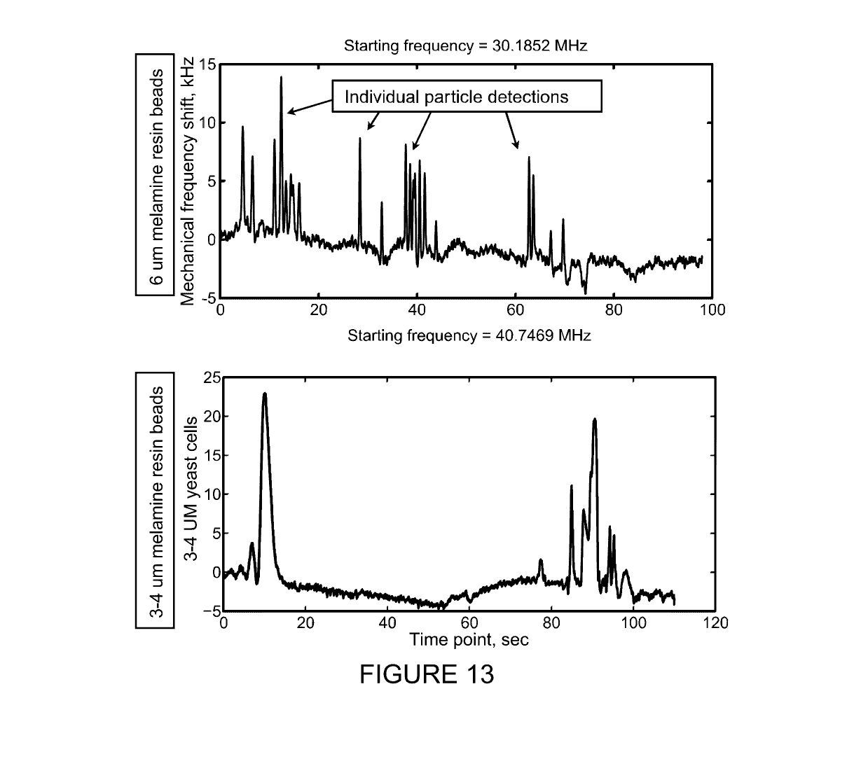

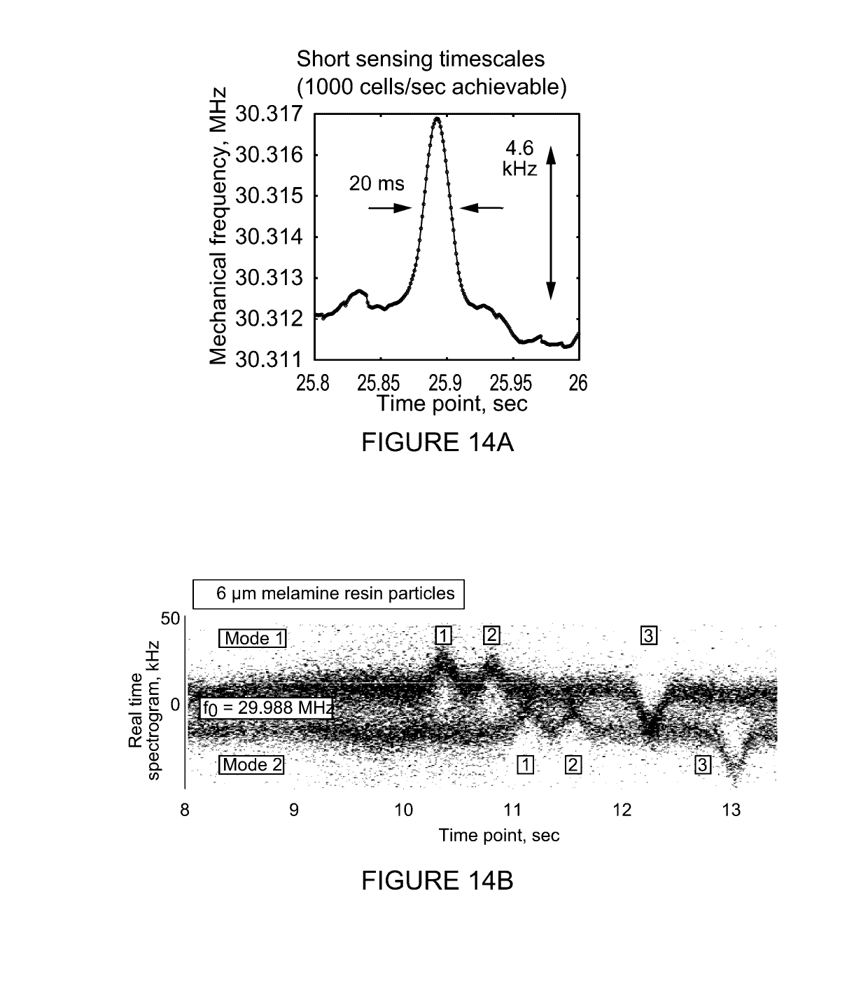

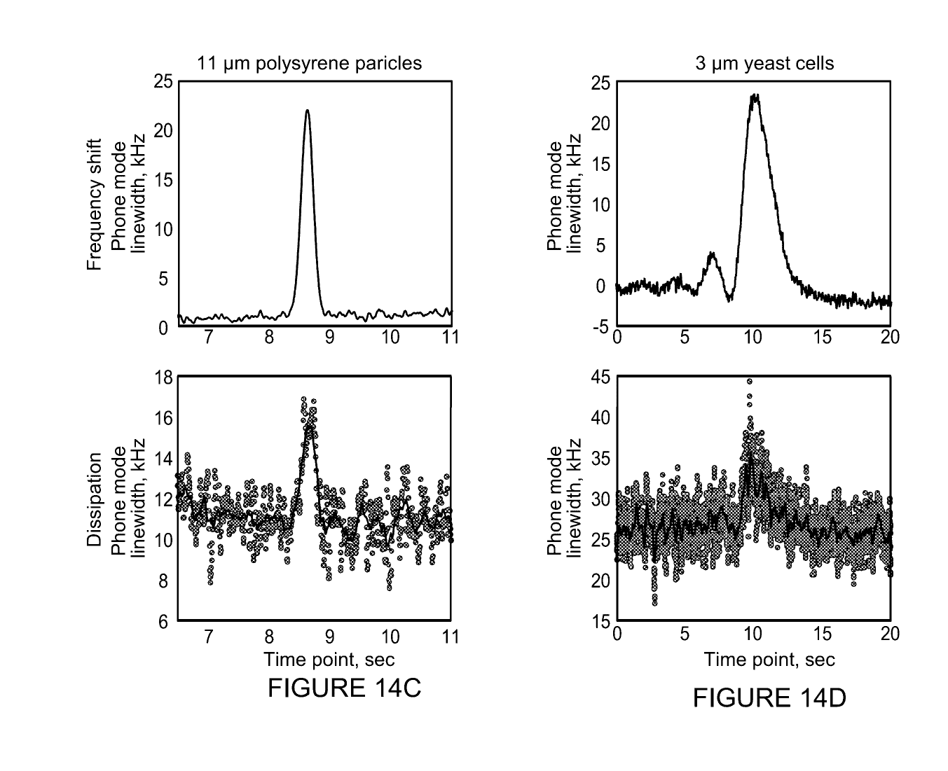

FIG. 12 is a schematic and FIGS. 13-14 are graphs illustrating an example real-time high-throughput sensing of particles flowing in a carrier liquid through the resonator. In FIG. 12, particles are flowed through the resonator device 10 with pressure provided by a syringe pump. A 1550 nm fiber-coupled laser light source 20 monitors the Brownian fluctuations of the resonator device 10 vibrational modes (e.g., about 30-50 MHz) with high sensitivity. Perturbation of the vibration frequency is captured using a real-time spectrum analyzer. In FIG. 13, example test particle include about 6 um, about 178 pg: Melamine resin particles, carboxylate modified, rhodamine B-marked, and about 3-4 um, about 37 pg: household yeast cells, rehydrated.

In FIG. 14A, a 20 ms timescale particle transit is illustrated, showing a measurement rate of 25 particles/second. An example sampling rate can reach about 1000 particles/second. The mechanical Q-factor limited theoretical maximum is around 10,000 particles/second.

In FIG. 14B, the wide spatial extent of the optical mode in bottle-shaped resonator device 10 enables simultaneous measurement of spatially separated phonon modes. FIG. 14B shows an example measurement of two frequency-adjacent modes that react to transiting particles with a time lag. Correlating the two spectra provides information on flow speed and spatial separation, with the potential to make two independent measurements of the same particle. Additionally, this also enables multimode sensing and inertial imaging capabilities with resonator device 10 devices.

In FIGS. 14C-D, the spectrum of scattered light also carries information on the mechanical dissipation associated with the particle. This can be gleaned from linewidth observations of the real-time optomechanical spectrum, where a greater linewidth indicates a greater phonon mode dissipation rate. FIGS. 14C-D present evidence of increased phonon dissipation during the transits previously shown in FIG. 3. The 30% increase of the phonon dissipation rate measured during the transit of an 11 micron size particle is more than an order of magnitude greater than would be estimated from the modification of the stored energy in the resonator by the particle. This implies that the increased dissipation is likely related to the viscoelastic properties of the particle material and boundary loss at the interface of the particle and the liquid.

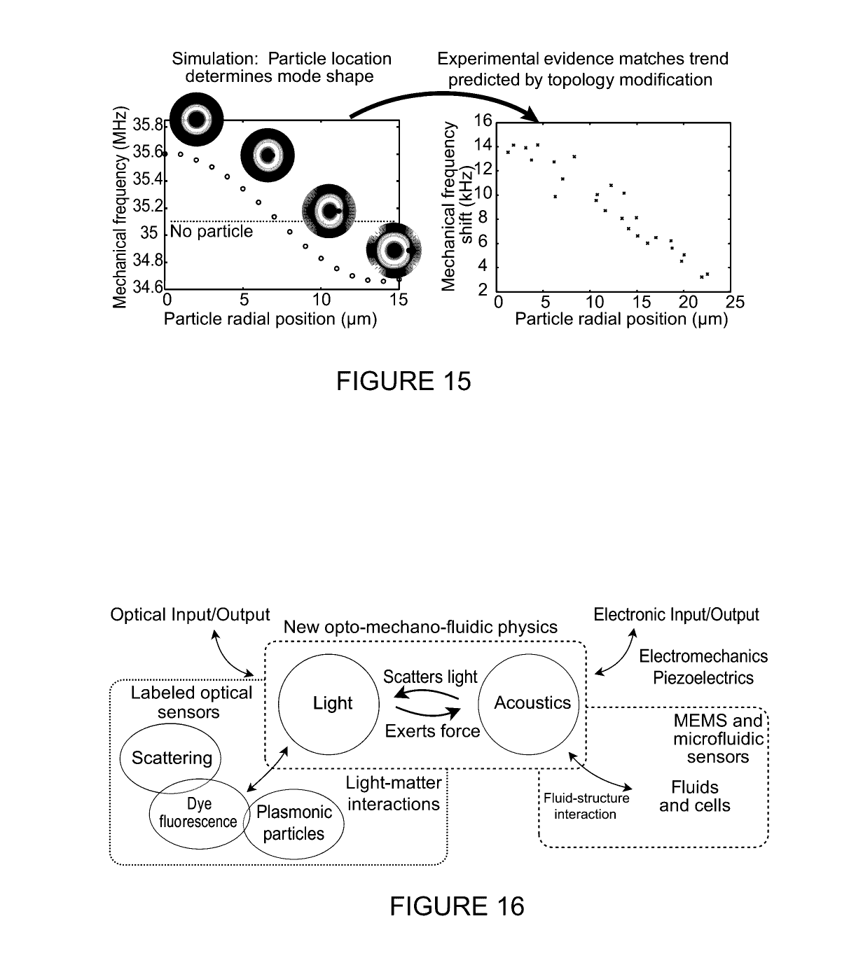

FIG. 15 is a graph of example vibration frequency shift corresponding to particle location that has been calculated and measured according to an acoustic mode topology (e.g. shape) particle sensing mechanism. According to the simulation on the left, the particle location determines mode, e.g., resonance, shape and or frequency. The measured example on the right matches trend predicted by topology modification. As the cell or other particle passes through the resonator device 10, the observed sensitivity is from a change of the acoustic mode topology in the presence of the cell or other particle, and the mechanism is sensitive to particle size, versus particle mass. For example, the sensing may also be provided by the mass loading mechanism.

FIG. 16 is a block diagram illustrating an example opto-mechano-fluidic physics. Resonator devices 10 can bridge between existing optical/acoustic biosensing modalities, and allow for extremely high rate viscometry, gravimetry, and flow cytometry. The vibrational sensing mechanism can potentially sense zero buoyant-mass particles. Resonator devices 10 can enable an acoustic flow cytometry which can be used with existing cytometry devices and/or other types of devices.

The systems, methods, devices, and logic described above may be implemented in many different ways in many different combinations of hardware, software or both hardware and software. For example, all or parts of the system may include circuitry in a controller, a microprocessor, or an application specified integrated circuit (ASIC), or may be implemented with discrete logic or components, or a combination of other types of analog or digital circuitry, combined on a single integrated circuit or distributed among multiple integrated circuits.

Many modifications and other embodiments set forth herein can come to mind to one skilled in the art having the benefit of the teachings presented in the foregoing descriptions and the associated drawings. Although specified terms are employed herein, they are used in a generic and descriptive sense only and not for purposes of limitation.

1. A system comprising, comprising:
a resonator device including walls and a channel formed by the walls, the channel configured to receive a liquid containing particles; and
where the walls are configured to simultaneously confine light and a mechanical or sound vibration, and when the liquid passes through the channel the liquid changes at least one of a resonance of the light and the mechanical or sound vibration based on a property of at least one of the liquid and the particles.

2. The system of claim 1, further comprising a light source to direct light through the resonator device.

3. The system of claim 2, where the light source comprises a laser.

4. The system of claim 1, where the resonator device is configured to provide label free detection of the particles or the liquid.

5. The system of claim 1, where the resonator device confines the light in an optical whispering gallery mode.

6. The system of claim 1, where the resonator device comprises a fused silica microfluidic and optomechanical device.

7. The system of claim 1, where thermal fluctuations and induced vibrations of a mechanical mode can modulate the light.

8. The system of claim 7, further comprising multiple mechanical modes that modulate the light to provide more information than one mechanical parameter.

9. The system of claim 1, where the resonator device mechanically entrains the liquid by shell oscillations.

10. The system of claim 1, where the optical modes and mechanical modes are simultaneously confined in the same region of the resonator device, enabling a high-degree of opto-mechanical interaction.

11. The system of claim 1, where the resonator device comprises an ultra-high-Q microfluidic opto-mechanical resonator.

12. The system of claim 1, where sensitivity of the resonator device from a mechanical effect provides measurement using an optical signal.

13. The system of claim 1, where sensitivity of the resonator device from an optical effect provides measurement using a mechanical vibration signal.

14. The system of claim 1, where the light and the vibration interact together through at least one of radiation pressure, electrostriction and scattering.

15. The system of claim 1, further comprising the resonator device being configured to provide measurement of a mechanical property of the liquid including at least one of bulk modulus, speed of sound, density, viscosity and non-Newtonian parameters.

16. The system of claim 1, further comprising the resonator device being configured to provide measurement of a mechanical property of particle including at least one of stiffness, volume, compressibility, speed of sound, mass and density

17. The system of claim 1, further comprising the resonator device being configured to provide measurement of an optical property of the liquid including at least one of a refractive index, optical absorption, scattering and luminescence.

18. The system of claim 1, where the walls comprise fused silica glass walls.

19. The system of claim 1, where the walls are generally cylindrical shaped.

20. The system of claim 1, where the channel includes a center portion that is wider in diameter than tapered end portions.

21. A method, comprising:
providing silica walls to form a channel;
flowing liquid and particles inside the channel;
simultaneously confining light and mechanical vibration in the channel while flowing the liquid and the particles; and
changing at least one of a resonance of the light and the mechanical vibration based on a property of the particles or the liquid as they flow inside the channel.

22. The method of claim 21, further comprising providing the light to the channel to measure a mechanical vibration effect.

23. The method of claim 21, further comprising providing the mechanical vibration to the channel to measure a light effect.
